(19) United States
(12) Patent Application Publication
(10) Pub. No.: US 2004/0039186 A1
(43) Pub. Date: Feb. 26, 2004

(54) Self-regulated apoptosis of inflammatory cells by gene therapy

(75) Inventors: Revati J. Tatake, Sandy Hook, CT (US); Steven D. Marlin, Sandy Hook, CT (US); Randall W. Barton, Farmington, CT (US)

Correspondence Address: BOEHRINGER INGELHEIM CORPORATION, 900 RIDGEBURY ROAD, P O BOX 368, RIDGEFIELD, CT, 06877, US

(73) Assignee: Boehringer Ingelheim Pharmaceuticals, Inc., Ridgefield, CT

(21) Appl. No.: 10/356,308

(22) Filed: Jan. 31, 2003

Related U.S. Application Data
(63) Continuation-in-part of application No. 09/032,297, filed on Feb. 27, 1998, now Pat. No. 6,525,184.
(60) Provisional application No. 60/039,266, filed on Feb. 28, 1997.

(51) Int. Cl. C07H 21/04; C12P 21/02; C07K 14/525; C12N 5/06; C12N 15/85

(52) U.S. Cl. 536/23.5; 435/455; 435/69.5; 435/320.1; 435/325; 530/351

(57) ABSTRACT

This invention relates to the therapeutic induction of supra-normal apoptosis in activated inflammatory cells, or cells at a site of inflammation, by introducing into those cells a chimeric gene containing an apoptosis-inducing gene (AIG) driven by a promoter of an inducible gene activated in inflammation and a promoter enhancer such that the inflammatory cells are targeted. In one embodiment, the chimeric gene comprises at least one TNFα promoter enhancer attached to a functional copy of a minimal TNFα promoter and further attached to at least one copy of an apoptosis-inducing gene, wherein expression of the gene is driven by the TNFα promoter. Examples of apoptosis-inducing genes include caspase-3, caspase-4, caspase-5, and Granzyme B. Advantageously, the TNFp-AIG chimeric gene is expressed in only those cells producing the inflammatory cytokine, TNFα. In addition, the TNFp-AIG chimeric gene also sequesters inducible TNFp transcription factors, thereby reducing endogenous production of TNFα. The invention also relates to methods of making and using self-regulated apoptosis chimeric genes that induce supra-normal apoptosis and pharmaceutical compositions containing them for treating inflammatory diseases.

### RELATED APPLICATION DATA

This application is a continuation-in-part of U.S. Ser. No. 09/032,297, filed Feb. 27, 1998 which claims, as does the present application, priority benefit of U.S. provisional application Serial No. 60/039,266, filed Feb. 28, 1997, the disclosures of all of which are incorporated by reference in their entireties.

### BACKGROUND OF THE INVENTION

This invention relates to the therapeutic induction of supra-normal apoptosis in inflammatory cells by introducing into those cells a gene which induces apoptosis (programmed cell death or non-necrotic cell death) in these cells. The Apoptosis-Inducing Gene (which will sometimes be referred to herein as AIG) is driven by a TNFα promoter (TNFp) or other inducible gene activated in inflammation. In one embodiment, apoptosis is selectively induced in those cells capable of producing TNFα. The TNFp-AIG or other chimeric gene may be conveniently introduced in vivo using conventional gene therapy techniques. Advantageously, in the embodiment wherein the chimeric gene is TNFp-AIG, it is expressed in only those cells producing the inflammatory cytokine, TNFα. In addition, since the TNFp-AIG chimeric gene contains the TNFα promoter elements, it also sequesters inducible, TNFp-selective transcription factors. Such sequestration results in a reduction in endogenous production of TNFα. The present invention relates specifically to TNFp-AIG and similar gene constructs, cells containing chimeric genes, methods for induction of apoptosis in cells transfected with chimeric genes, pharmaceutical compositions containing chimeric genes, methods for in vitro selection of TNFα non-producer somatic cell variants within a TNFα producing cell population and the like, a method for identifying dominant negative/dominant suppressive genes responsible for inhibiting TNFα production and therapeutic methods using the chimeric gene.

In many inflammatory conditions, cytokines such as IL-1, IL-10, GM-CSF and TNFα are excessively produced as a result of mass aggregation and accumulation of inflammatory cells (Brennan F. M. et al., British Medical Bulletin 1995, 51/2, 368-384). Upregulation and/or dysregulation of cytokines in inflamed tissue may be directly or indirectly responsible for exacerbation of chronic inflammatory diseases. For example, the most marked pathology in rheumatoid arthritis (RA) is displayed at the local site of inflammation (i.e., the synovial joints). Therefore, it is likely that the cytokines produced in the synovial joints of RA patients play an important role in the disease process. Of those cytokines, IL-1 and TNFα are believed to be responsible for the devastating cartilage destruction and bone erosion which characterizes RA (Dayer J. M. et al., J. Exp. Med., 1985, 162, 1208-1215; Gowen M. et al., Nature, 1983, 306, 378-380). The presence of excessive amounts of IL-1 and TNFα in the synovial joints has been shown to accelerate development of collagen-induced arthritis in rodents (Brennan F. M., et al., Clin. Expt. Immunol., 1994, 97/1, 1-3). Excessive amounts of TNFα and IL-1 are produced in the synovial tissue by a variety of cell types at the cartilage-pannus junction, including cells of the macrophage lineage, macrophage-like synoviocytes, activated T-cells and possibly fibroblast-like synoviocytes (Chu C. Q. et al., Arthritis & Rheumatism, 1991, 34, 1125-1132; Deleuran B. W., et al., Arthritis & Rheumatism, 1992, 35, 1170-1178).

In addition to the above described inflammatory effects, TNFα plays a ubiquitous and key role in a variety of pro-inflammatory events, such as induction of IL-1 activity in monocytes. Indeed, anti-TNFα neutralizing antibodies have been shown to reduce overall IL-1 production (Portillo, et al., Immunol., 1989, 66, 170-175; Brennan F. M., et al., British Medical Bulletin 1995, 51/2, 368-384). Thus, an added benefit to blocking the effect of the inflammatory cytokine TNFα is the reduction in production of the equally destructive pro-inflammatory mediator, IL-1. Furthermore, it is well known that TNFα is a transcriptional activator of other inflammation-related genes. For example, the presence of TNFα stimulates production of other cytokines (such as GM-CSF) and cell surface receptors, including HLA class II antigens and adhesion molecules (Alvaro-Garcia J. M., et al., J. Exp. Med., 1989, 146, 865-875), which results in continuous recruitment of activated T cells and neutrophils resulting in synovial inflammation and hyperplasia and ultimately, in augmented destruction of cartilage and bone (Allen J. B., J. Exp. Med., 1990, 171, 231).

Conventional therapy against inflammatory disorders is typically directed against symptomatic inflammation. Such therapies provide only temporary relief without significantly delaying disease progression. In contrast, therapies targeting TNFα and other factors induced in the inflammatory process are likely to be more promising. For example, in collagen-induced arthritis animal models, an anti-TNFα antibody and soluble TNFα receptor-IgG chimera effectively reduced paw swelling, joint involvement and cartilage and bone destruction (Williams R. O. et al., Proc. Natl. Acad. Sci., 1992, 89, 9784-9788). Human trials using both humanized anti-TNFα antibodies and TNFα receptor-IgG chimeric molecules produced dramatic results (Elliott M. J., et al., Arthritis and Rheumatism, 1993, 36, 1681-1690; Elliott M. J., et al., Lancet, 343, 1105-1110). Although treatment with these TNFα antagonists appears to be well tolerated, it also results in production of antibodies against the recombinant proteins. Thus, these therapies may not be suitable for long term treatment and do not achieve true disease abatement. In order to actually modify progression of the disease, TNFα must be continuously targeted using TNFα specific therapies. Such a therapeutic protocol is impractical with these biologic agents and would be difficult to administer in the long term.

In an alternate therapeutic option, inflamed synovium may be removed using surgical synovectomy (Herold N. and Schroder H. A., Acta Orthop. Scand., 1995, 66, 252-254; Ogilvie-Harris D. J. and Weisleder L., Arthroscopy, 1995, 11, 91-95), chemical (Cruz-Esteban C. and Wilke W. S., Bailliere's Clinical Rheumatol., 1995, 9, 787-801) or radiation-induced synovectomy (Cruz-Esteban C. and Wilke W. S., Bailliere's Clinical Rheumatol., 1995, 9, 787-801). Marginal to good results follow arthroscopic surgery. Non-surgical synovectomy is performed using various chemical agents such as osmic acid, alkylating agents such as nitrogen mustard and thiotepa, methotrexate. Unfortunately, non-surgical synovectomies (including chemical and radiation-induced) are procedurally complicated, provide only short term relief and show only patchy reduction of the synovial hyperplasia. Furthermore, most of the non-surgical alternatives are potential teratogens. Moreover, tissue damage arising from chemical or surgical intervention often results in an inflammatory response. Finally, it should be noted that these approaches suffer from the risks and side-effects commonly associated with conventional pharmaceutical therapy and invasive surgical procedures, including the expense and inconvenience of hospitalization and rehabilitation.

Accordingly, a need still exists for an effective therapeutic approach to treating inflammatory disorders in general and RA in particular.

### SUMMARY OF THE INVENTION

This invention overcomes the drawbacks associated with previous therapies for treating inflammatory disorders by providing a novel therapeutic approach. The present application is based, in part, on Applicants' discovery of chimeric genes that can induce supra-normal apoptosis. According to one embodiment of this invention, supra-normal apoptosis is selectively induced in TNFα-producing inflammatory cells, causing destruction of these cells without an associated inflammatory reaction.

One objective of this invention is to provide a therapeutic method comprising the step of introducing into the inflammatory cells of a mammal, or cells at a site of inflammation, a chimeric gene containing a self-regulating apoptosis-inducing gene (AIG). The AIG is driven by a promoter such as a TNFα promoter (TNFp; see FIGS. 1 and 2), and, preferably, a promoter enhancer. Therefore, it is expressed in all and only those cells capable of producing TNFα.

Another objective of this invention is to provide TNFp-AIG and the like chimeric gene constructs, processes for making them, methods of using them, and preparations containing them.

Yet a further objective of this invention is to provide a method for the induction of apoptosis in cells transfected with the TNFp-AIG chimeric gene, a method for the in vitro selection of TNFα non-producer somatic cell variants in a population, a method for identifying dominant/negative genes responsible for the genesis of a TNFα non-producing population and a method for identifying products responsible for regulation of TNFα production (FIG. 10).

These and other objectives will be readily appreciated by those of ordinary skill in the art based upon the following detailed disclosure of the invention.

### BRIEF DESCRIPTION OF THE FIGURES

FIG. 1 is a schematic representation of TNFp-AIG chimeric genes of this invention. Apoptosis Inducing Gene (AIG) could be any, but not limited to, the genes listed, viz., caspases 1 to 10, Granzyme B, FasLigand, etc.

FIG. 2 is a schematic drawing depicting the results of gene therapy using TNFp-AIG chimeric genes of this invention.

FIG. 3 is a summary of deletion constructs used for identification of the inducible cis elements of the TNFα promoter using luciferase gene (Luc) expression as the reporter system.

FIGS. 4(a and b) provide a summary of results obtained using the constructs described in FIG. 3. Transient expression of the constructs was assessed in two different TNFα-producing cell lines, viz., Jurkat (FIG. 4a) and THP-1 (FIG. 4b). Histograms in each Fig. show stimulation index as a measure of inducibility by activating agents such as PMA (FIG. 4a) or LPS (FIG. 4b) for individual experiments. The line superimposed in each Figure indicates the mean inducibility averaged from 4 to 6 experiments.

FIG. 5 is a flow chart for preparation of the TNFpAIG using selected native elements of the TNFα promoter and prodomain-deleted AIGs (AIGs used are caspase 3 and caspase 4/5).

FIGS. 6(a, b, and c) provide a summary of results from representative experiments performed to see expression of the chimeric TNFpAIGs. Apoptosis in transiently-transfected Jurkat cells (FIGS. 6a and 6b) and THP-1 (FIG. 6c) cells was assessed using Cell Death Elisa (CDE assay). In all three Figures, histograms with black shading represent transfection control, where cells were treated with the transfecting agent in the absence of DNA. Histograms with diagonal shading represent the TNFp elements driving expression of the luciferase gene and solid histograms represent the same TNFp elements driving expression of either AIG.1 or AIG.2. The number in parenthesis above the solid histograms represent enrichment factor indicative of supra-normal apoptosis (ratio of apoptosis induced by TNFpAIG to the TNFpLuc control vector).

FIGS. 7(a and b) is a diagrammatic representation of a TNFp-AIG chimeric gene of this invention, comprising multiple copies of the inducible cis elements of the TNFα promoter which, in turn, drive expression of the AIG (FIG. 7a). A diagrammatic representation of a TNFpAIG chimeric gene, comprising multiple copies of the inducible cis elements of the TNFα promoter, driving expression of the AIG, downstream of which is a 3′ untranslated region of the TNFα gene (TNF3′UTR) (FIG. 7b). The 3′UTR of the TNFα gene is implicated in the regulation of the inducible expression of TNFα (Han, J., et al., J. Immunology, 1991, 146, 1843-1843, Crawford, E. K., et al., J. Biol. Chem., 1997, 272, 21120-21137, and FIG. 9).

FIGS. 8(a and b) are flow charts of schemes for preparing TNFα super-promoter-AIG chimeric constructs.

FIG. 9 shows a summary of the results of two experiments to show the regulatory effect of the TNF3′UTR on inducible expression of the luciferase reporter gene. The transient transfection was performed in a fibroblast cell line. Dotted histograms represent inducibility of TNFpLuc in the absence of TNF3′UTR and solid white histograms represent inducibility of TNFpLuc in the presence of TNF3′UTR. Similar results are obtained in the Jurkat cell.

FIG. 10 is a diagrammatic representation for the selection of TNFα non-producer somatic cell variants within a TNFα-producing cell population and identification of dominant negative suppressive genes responsible for inhibiting TNFα production.

FIG. 11 shows cleavage of a caspase-3-selective substrate, Z-DEVD-AFC, with J-E6 cells transfected with cDNA encoding AIG.1. J-E6 cells were transfected with a control vector (cDNA) or the vector expressing AIG.1. This assay indicates activation of caspase-3 as a measure of apoptosis. Transfected cells were harvested at various times after transfection. The cells were lysed and the caspase-3 activity, indicative of functionally active expression of AIG.1, was assessed by determining release of AFC from Z-DEVD-AFC. AIG-transfected cells exhibited approximately three times higher activation of caspase-3, indicative of supra-normal apoptosis.

FIG. 12 shows cleavage of endogenous Poly (ADP) ribose polymerase (PARP) by cells expressing AIG.1. HeLa cells were transfected with AIG.1 and a control vector and harvested 24 hrs later for detection of PARP cleavage. AIG-transfected cells exhibited PARP cleavage, indicating induction of supra-normal apoptosis.

FIG. 13 shows induction of apoptosis in Jurkat (FIG. 13a) or THP-1 cells (FIG. 13b). Cells were transfected with the chimeric genes −1005AIG.1/2 and −1005AIG.1/2 3′UTR. The cells were harvested after 24 hours, and DNA fragmentation was assessed by cell death ELISA (CDE). These experiments showed supra-normal apoptosis in cells transfected with chimeric genes.

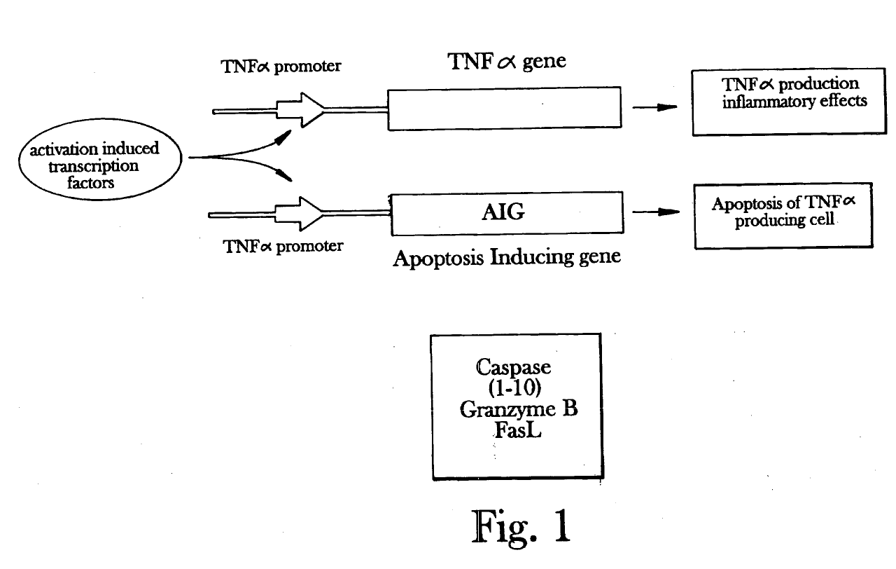

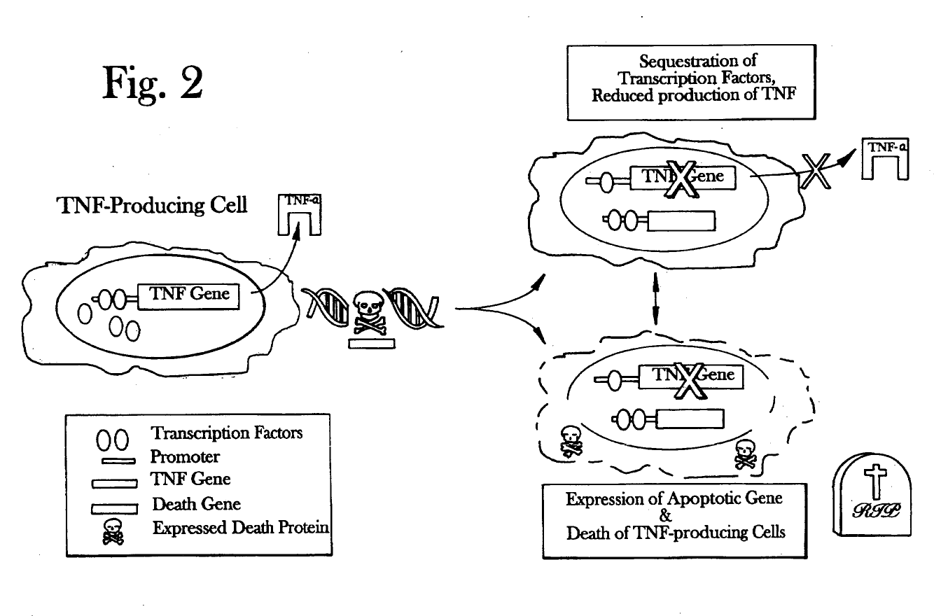

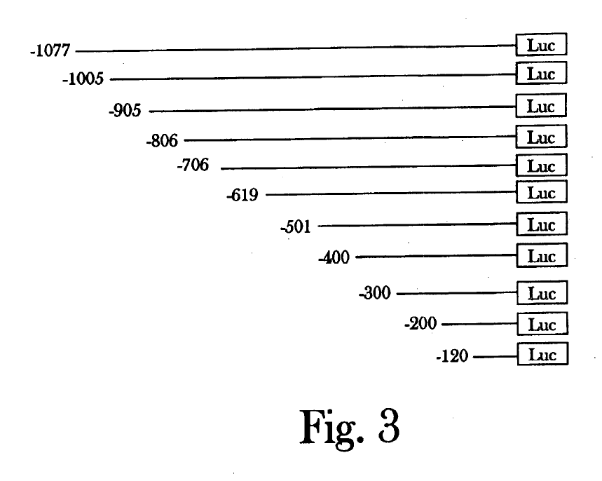

### DETAILED DESCRIPTION OF THE INVENTION

The invention described herein overcomes the drawbacks in the field by providing novel chimeric nucleic acid molecules for use in therapeutic compositions and methods of using such compositions. The compositions are directed to selectively induce apoptosis in TNFα producing cells causing destruction of these cells.

As used herein, the abbreviation 3′UTR means 3′ untranslated region.

The abbreviation “AIG” refers to Apoptosis Inducing Gene. An AIG includes Granzyme B.

The abbreviation “CsA” refers to Cyclosporin A, a biologically active fungal metabolite with immunosuppressive properties.

The abbreviation “DN” refers to dominant/negative gene products which have negative affect on expression or function of other genes or gene products.

The abbreviation “ER” refers to Enhancer Region, whereby ER1 has SEQ ID NO: 10, ER2 has SEQ ID NO: 12.

The abbreviation “GB” refers to Granzyme B.

The abbreviation “PMA” refers to Phorbol Myristate Acetate.

The abbreviation “RA” refers to Rheumatoid Arthritis.

The abbreviation “TNFα” refers to tumor necrosis factor alpha.

The terms “TNF promoter”, “TNFα promoter” and “TNFp” are used interchangeably herein. Unless noted to the contrary, these terms refer to the entire nucleotide sequence corresponding to a native TNFα minimal promoter sequence attached to one or more upstream enhancer elements, either present naturally, native, or genetically engineered in the laboratory.

Amino acid “substitutions” are defined as having one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

“Conservative variants” refer to the substitutions of amino acids in a polypeptide.

“Allelic variants” refer to the variation at the nucleic acid and protein level either due to conservative or non-conservative substitutions giving rise to alternative form of the same gene.

“Reporter” molecules are chemical moieties used for labeling a nucleic acid or amino acid sequence. They include, but are not limited to, radionuclides, enzymes, fluorescent, chemiluminescent, or chromogenic agents. Reporter molecules associate with, establish the presence of, and may allow quantification of a particular nucleic or amino acid sequence.

“Reporter genes” are nucleic acids encoding a functional protein, such as luciferase, that may be used to assess the activity of heterologous promoters.

A “functional fragment” of a polynucleotide or nucleic acid comprises all or any part of the nucleotide sequence having fewer nucleotides, which can be used as genetic material sufficient to initiate transcription of a gene or encode for a functional subunit of a polypeptide.

“Normal apoptosis” as used herein relates to a default process, in which a cell undergoes natural, albeit programmed cell death (PCD), due to absence of external stimuli for its survival. This default process of PCD can be impaired due to a defect in expression of one or more genes regulating PCD. Examples of genes regulating PCD are p53, Bcl-2, Bcl-X, etc. Normal apoptosis can be restored in the cells impaired for PCD by complementing the impaired cells with a non-defective version of the respective gene(s).

“Supra-normal apoptosis” relates to a non-natural process, in which cells are forced to undergo apoptosis even in the presence of external stimuli for their survival. Supra-normal apoptosis is induced in cells by supplementing them with an “effector” mechanism (e.g. genes, proteins, chemicals or radiation), resulting in forced demise of cells. Examples of genes or proteins that can be used to induce supra-normal apoptosis are various caspases involved in apoptosis cascade, Granzyme-B, etc. Exposing cells to chemicals such as ceramide or UV radiation can also result in supra-normal apoptosis.

This invention is based upon evidence that apoptosis of inflammatory cells in certain inflammatory diseases is therapeutically beneficial. The invention specifically relates to self-regulated apoptosis by gene therapy. Broadly speaking, in the practice of the invention, a chimeric gene comprising at least one promoter enhancer attached to at least one functional copy of a minimal promoter, the promoter being a gene or combination of genes activated in inflammatory cells or in cells at a site of inflammation, is attached to at least one copy of an apoptosis-inducing gene (AIG), such that the expression of the apoptosis-inducing gene is driven by the promoter, thus targeting the inflammatory cells. Example promoters of inducible genes activated in inflammation include, but are not limited to, cytokines, interleukins and their receptors, cell adhesion molecules and their ligands, chemokines and their receptors, pro-inflammatory enzymes, and the like. Chimeric genes according to the invention comprise enhancer, promoter, and AIG elements in direct, distal, or proximal attachment, and combinations thereof. As mentioned above and will be discussed in more detail below, in some embodiments, multiple copies of the enhancer, promoter, and/or AIG are employed for maximal efficacy.

In order that the invention herein described may be more fully understood, the following detailed description is set forth, with emphasis on chimeric genes comprising at least one TNFα promoter enhancer attached to at least one functional copy of a minimal TNFα promoter and further attached to at least one copy of an AIG for illustrative purposes only. Though the examples that follow also employ these types of constructions, it will be appreciated by skilled workers that the basic constructs described herein may be altered to provide other embodiments that utilize products, processes, methods, and compositions of the invention with other promoters comprising inducible genes activated in inflammation such as the types listed above that exhibit similar functions that can be used to target cells at the site of infection.

For example, cytokines and interleukins useful as promoters in the construction of chimeric genes of the invention include, but are not limited to, TNFα, TNFβ, IL-1α, IL-1β, IL-2, Il-6, IL-9, GM-CSF, interferon-γ, and the like, and functional fragments and mixtures thereof. Cell adhesion molecules and their ligands include, but are not limited to, selectins, integrins, and members of the immunoglobulin superfamily such as ICAM-1, V-CAM, and the like, and functional fragments and variants and mixtures thereof. Chemokines and their receptors include, but are not limited to, the C-X-C and C-C family members such as MIP-1α, MIP-1β, MCP1-4, RANTES, Mig, NAP2, IP10, Gro α-γ and the like, and functional fragments and variants and mixtures thereof. Pro-inflammatory enzymes include, but are not limited to COX-2, iNOS, phospholipases, proteases (including matrix metalloproteases), and the like and functional fragments and mixtures thereof.

To clarify the discussion below of exemplary TNFp-AIG chimeric genes of this invention, the following sequences are illustrated:

SEQ ID NO: 1 is the nucleotide sequence corresponding to the full-length, reference human TNFα promoter sequence, as published in (Takashiba S., et al., Gene, 1993, 131, 307-308). Nucleotide numbers used herein refer to the numbering of this sequence.

SEQ ID NO: 2 is the native TNFα promoter sequence of the gene that was used in this invention (−1077 nucleotides from the transcription start site, TSS). There are a few differences in the sequence of the TNFp in SEQ ID NO: 1 and SEQ ID NO: 2. Such differences in the nucleotide sequences of the TNFα promoter have been reported (Takashiba S., et al., Gene, 1993, 131,307-308).

SEQ ID NO: 3 is the native minimal TNFα promoter sequence (nucleotide −120 through −TSS, which includes at least one enhancer element (k1 site; see Pauli, U., Crit. Rev. in Eukaryotic Gene Expression, 1994, 4, 323-344; Rhoades K. L., et al., J. Biol. Chem., 1992, 267, 22102-22107; and Takashiba S., et al., Gene, 131, 307-108).

SEQ ID NO: 4 is the chimeric gene TNFp120 AIG.1 (containing −120 TNFp driving the expression of the prodomain-deleted variant of CPP32 gene (caspase 3, published Tewari M. et al., Cell, 1995, 81(5), 801-809, with the variation being V239A).

SEQ ID NO: 5 is the chimeric gene TNFp706 AIG.1 (containing −706TNFp driving expression of the prodomain-deleted CPP 32 gene.

SEQ ID NO: 6 is the TNFp1005 AIG.1 (containing −1005 TNFp driving expression of the prodomain-deleted CPP 32 gene).

SEQ ID NO: 7 is the chimeric gene TNFp120 AIG.2 (containing −120 TNFp driving expression of the prodomain-deleted Ty/x gene. (Sequences of Ty (caspase 5) and Tx (caspase 4) genes are published in the ref. Faucheu, C., et. al., Eur. J. Biochem., 236, 207-213, 1996; Faucheu, C., et. al. EMBO J., 14, 1914-1922,1995).

SEQ ID NO: 8 is the chimeric gene TNFp706 AIG.2 (containing −706TNFp driving expression of the prodomain-deleted Ty/x gene.

SEQ ID NO: 9 is the TNFp1005 AIG.1 (containing −1005 TNFp driving expression of the prodomain-deleted Ty/x gene).

SEQ ID NO: 10 is the enhancer region 1 (ER1) of the TNFα promoter encompassing nucleotides −1005 to −905.

SEQ ID NO: 11 is the enhancer region 2 (ER2) of the TNFα promoter encompassing nucleotides −706 to −517.

SEQ ID NO: 12 is additional multiple cloning sites (MCS) genetically engineered upstream of the −120 minimal TNFα promoter in the −120pGL3 construct.

SEQ ID NO: 13 is the 3′ untranslated region (3′UTR) of the TNFα gene (Nedwin, G. E., et al., Nucleic Acid Research, 1985, 13, 6361-6373).

The elements of the TNFα promoter for preparation of chimeric gene constructs according to this invention are selected from elements which are capable of inducing expression of a therapeutic gene driven by the TNFα promoter. These promoter elements will be referred to herein as “inducible cis elements”, “cis-inducible elements” or “enhancer elements” of the TNFα promoter.

The enhancer elements may be physically linked to the minimal promoter sequence, or separated from the minimal promoter by a linker sequence which may or may not have unique restriction sites. Thus, as summarized above, enhancer elements may be attached directly, distally, proximally, or any combination thereof, to chimeric genes of the invention. These are typically constructed upstream of the promoter. Examples of TNFα enhancer elements are set out in SEQ ID NO: 10 and SEQ ID NO: 11. Alternatively, functional fragments or variants and combinations thereof may be employed. Some preferred gene constructs according to this invention include those that have multiple copies of the enhancer elements, i.e., 2 or more copies. Some embodiments have about 2 to 25, more narrowly 2 to 10, and even more narrowly, 2 to 5 copies.

The terms “TNF promoter”, “TNFα promoter” and “TNFp” are used interchangeably herein. Unless noted to the contrary, these terms refer to the entire nucleotide sequence corresponding to a native TNFα minimal promoter sequence attached to one or more upstream enhancer elements (either present naturally i.e. native, or genetically engineered in the laboratory). Examples include, but are not limited to: SEQ ID NO: 1, SEQ ID NO: 2, and SEQ ID NO: 3, and functional fragments, variants, and mixtures of any of these. Many functional fragments and variants of these TNFα sequences and others described herein share a sequence homology of at least about 80%, and in some cases over 90%, to their native and genetically engineered counterparts, but these are known to skilled workers and defined in the references cited herein. Many functional fragments and variants of these TNFα sequences and others described herein share a sequence identity of at least about 80% and in some cases over 90%, to their native and genetically engineered counterparts, but these are known to skilled workers and defined in the references cited herein. Various alignment programs known in the art can be used to determine sequence homology or identity including but not limited to the ALIGN alignment program.

Any apoptosis-inducing gene may be used in the chimeric genes and methods described herein. The apoptosis inducing gene used for the chimeric therapeutic genes of this invention can be the same or different from the type of apoptosis inducing gene present in the native sequence of the TNFα-producing inflammatory cells (if those cells naturally contain an apoptotic gene). Preferred AIGs include, but are not limited to, members of the ICE/CED3 family of apoptosis inducing proteases (such as caspase-1 (ICE), hICE, ICE-LAP45, Mch2α), caspase-2 (ICH1), caspase-3 (CPP32, Yama, Apopain), caspase-4 (TX, ICH2, ICE rel II), caspase-5 (ICE rel III, TY), caspase-6 (Mch-2), caspase-7 (Mch-3, ICE-LAP3, CMH-1), caspase-8 (MACH, FLICE, Mch-5), caspase-9 (ICE-LAP6, Mch6) and caspase-10 (Mch4)), members of the granzyme family (such as Granzyme A and Granzyme B), Fas ligand (FasL), and functional fragments, variants, and mixtures of any of these. Some embodiments employ caspase 3, caspase 4, caspase 5, Granzyme B, and functional fragments, variants, and mixtures thereof. With the exception of FasL, these genes, when over-expressed following transfection, induce apoptosis in the transfected cells (Miura M., et al., Cell, 1993, 75, 653-660; Chinnayan A. M., et al., Cell, 1995, 81, 505-512; Los, et al., Nature, 1995, 375, 81; Muzio, et al., Cell, 1996, 85, 817-827).

In the case of FasL, apoptosis is induced (either in an autocrine or a paracrine fashion) in only those cells that express Fas receptor. Therefore, the TNFp-FasL chimeric gene construct offers a second level of selectivity. Another advantage of the TNFp-FasL chimeric gene is the selective targeting of those disease-producing cells in the synovium that do not express TNFα (thereby failing to drive expression of the apoptosis inducing gene), but do express Fas receptor on the surface. In this case, FasL will be expressed by the cells that are capable of producing TNFα such as activated macrophages and T cells. These cells will then induce apoptosis in Fas-expressing cells such as hazardous activated T cells and Fas-expressing synoviocytes.

This invention provides a novel therapeutic method comprising the step of introducing into the cells of a mammal a chimeric gene comprising an apoptosis-inducing gene (AIG) driven by the TNFα promoter (TNFp). Examples of chimeric genes of the invention are set out in SEQ ID NOs: 4, 5, 6, 7, 8, and 9. Alternatively, functional fragments or variants of these may also be employed. Without wishing to be bound by theory, as a result of being controlled by the TNFp, AIG is expressed in only those cells producing the inflammatory cytokine, TNFα. Therefore, any cells expressing TNFα will be self-destructive, while cells that do not express TNFα will be unaffected. Advantageously, this methodology can target any TNFα-producing cells (such as activated macrophages, activated T-cells and macrophage-like and possibly fibroblast-like synoviocytes) without regard to cell type. Indeed, the targeted TNFα-producing cell can be one which normally does or normally does not carry or expresses an apoptosis gene in its native, unaltered form. Therefore, using the chimeric genes and methods of this invention, the cellular sources of TNFα can be destroyed in a highly selective manner.

Another advantage of using the TNFp-AIG chimeric gene of this invention is that TNFp sequesters transcription factors needed by endogenous TNFp, thereby leading to a reduction in endogenous TNFα production. In one preferred embodiment, TNFp is present in the therapeutically targeted cell in vast excess. This may be accomplished by introducing multiple copies of the transfected gene into the cell. Alternatively, the TNFp-AIG chimeric gene according to this invention can contain multiple copies of the inducible cis elements of the TNFα promoter. As mentioned above, multiple copies of the “inducible enhancer elements” of TNFp are present in some embodiments of the TNFp-AIG chimeric genes of this invention. By including multiple copies of the inducible cis elements of the TNFp construct, the transcriptional factors needed by the transfected cell to produce TNFα are sequestered by the exogenously introduced sequence. This preferred chimeric TNFp-AIG construct is characterized by an increased effectiveness in competing for the TNFp-specific transcription factors as compared to chimeric genes of this invention containing only a single enhancer element linked to TNFp. The “inducible super promoter” constructed in this way is capable of: (1) more effectively competing for TNFα specific inducible transcription factors and (2) driving expression of the apoptosis inducing gene in an augmented fashion by virtue of multiple enhancing elements.

For example, in rheumatoid arthritis patients, synovectomy, i.e., removal of synovial tissue, has been shown to be clinically beneficial. Unlike conventional and surgical synovectomy procedures, the cell-targeted therapeutic method described herein targets only cells producing TNFα. Thus, advantageously, the introduction and expression of the TNFp-AIG chimeric gene, and subsequent therapeutic induction of supra-normal apoptosis does not induce an inflammatory response. Accordingly, methods of this invention are comparatively selective and result in minimal tissue damage and a reduction in inflammation.

The products and methods described herein are useful for the treatment of other inflammatory disorders as well. Such inflammatory disorders include, but are not limited to, multiple sclerosis, Guillain-Barre syndrome, Crohn's disease, ulcerative colitis, psoriasis, graft versus host disease, lupus erythematosus, insulin-dependent diabetes mellitus, psoriatic arthritis, sarcoidosis, hypersensitivity pneumonitis, ankylosing spondylitis and related spondyloarthropathies, Reiter's syndrome and systemic sclerosis. Thus, this invention encompasses methods for treating an inflammatory disorder in a patient by inducing supra-normal apoptosis in inflammatory cells or cells at a site of inflammation of the patient by introducing into the cells at least one chimeric gene of the invention. This is typically accomplished by preparing a pharmaceutical composition containing at least one chimeric gene of the invention and typically a pharmaceutically acceptable carrier, and administering the composition to a patient using standard means. In some embodiments, the pharmaceutical composition is delivered directly to the site of inflammation using local topical, intravenous, intraperitoneal, and similar methods. Further methodology is discussed below.

In addition to the therapeutic indications, the genes and cells according to this invention can be used in a variety of useful screening and selection methods. In one such method, TNFα non-producer somatic cell variants within a TNFα producing cell population can be selected in vitro by introducing a TNFp-AIG chimeric gene into the TNFα producing cell population. Cells producing TNFα will undergo apoptosis. Cells that do not produce TNFα will survive. Selection of those cell variants possessing the survival phenotype is an easy way to identify TNFα non-producer cells. Such a selection process may be used to determine expression of genes that act in-trans to regulate activity of the TNFα promoter, thereby reducing TNFα production. Such genes are characterized as dominant negative (DN)/dominant suppressive genes in other systems (Behrends S., et al., J. Biol. Chem. 1995, 270, 21109-21113; Zhang S., et al., J. Biol. Chem., 1995, 270, 23934-23936; Watowich S. S., et al., Mol. Cell Biol., 1994, 14/6, 3535-3549).

In a further in vitro method, a TNFp-AIG chimeric gene according to this invention can be used to identify dominant negative genes responsible for the genesis of a TNFα non-producing cell population. According to this method, a TNFp-AIG chimeric gene according to this invention is introduced into cells that produce TNFα. Barring the presence of a dominant negative gene, those cells should undergo apoptosis upon activation. Therefore, it can be deduced that surviving variants possess a dominant negative gene capable of down-regulating TNFα production. The dominant negative gene can be readily identified by producing a cDNA library and transfecting cell lines (e.g., Jurkat and THP-1). These cells are either stable transfectants of an inducible TNFp-AIG chimeric gene or TNFp-luciferase gene TNFp-AIG transfected cells will be selected for the survival phenotype following in vitro activation; survival phenotype is indicative of the effect of the DN genes. In the cells transfected with TNFp-luciferase gene, reduction in the luciferase activity will be indicative of the DN gene effect. Dominant negative genes identified using this protocol can be used as the future therapeutic agents themselves. Such genes will be the candidates for gene therapy in order to reduce TNFα production.

The methods utilized for gene transfer are grouped into two broad categories:

1. Direct approach: In situ transduction of the therapeutic gene into target cells such as synoviocytes using a suitable vector as a carrier for the therapeutic gene. The vector containing therapeutic gene is injected directly into the affected area (e.g., an arthritic joint).

2. Indirect approach: Ex-vivo transfection of the therapeutic gene into target cells such as synoviocytes. In this approach, the synovium is removed from joints, synoviocytes are isolated and cultured in vitro. In vitro cultured cells are transfected with the therapeutic gene, and genetically modified synoviocytes are transplanted back into the synovium.

For in vivo transfer, several vectors have been evaluated for their efficacy in gene delivery (Nita, et al., Arthritis & Rheumatism, 1996, 39/5, 820-828). Among the vectors used for gene therapy, the vectors derived from retroviruses are by far the best developed. They are able to insert genetic material in the host genome and produce stable transfectants. These vectors, however, are unable to infect non-dividing cells and, since they are inserted in the host genome, the possibility of insertional mutagenesis cannot be ruled out. In comparison, the vectors derived from adenoviruses infect dividing as well as non-dividing cells and deliver DNA episomally. The disadvantage of adenovirus based vectors is that these vectors continue to produce viral proteins in infected cells making them potentially antigenic. A third type of viral based vectors is derived from Herpes simplex viruses (HSV), which are also capable of infecting dividing as well as non dividing cells.

Among the non-viral vector systems, cationic liposomes and naked plasmid DNA have been evaluated. Liposomes are at the most advanced stage of development, although certain types of cells such as muscle and skin take up, retain and express naked plasmid DNA.

Particle-mediated gene-delivery system is also possible (Rakhmilevich, et al., PNAS, 1996, 93, 6291) and is a promising approach.

The following “in vivo” gene delivery protocols can be, used to deliver the chimeric genes of this invention:

(1) Nita et al., Arthritis and Rheumatism, 1996, 39, 820-823

In Vivo Experiment in Rabbits:

Each vector is injected intra-articularly into 1 knee joint. For viral vectors, between 10⁸ and 10⁹ particles suspended in 0.5 ml balance salt solution are injected per knee.

Liposome-DNA complexes (200 nmoles of DC-Chol complexed with 20 μg of DNA/ml) in 1 ml balance salt solution are injected per knee.

(2) Methods in Molecular Medicine: Gene Therapy Protocols, Paul Robbins, ed., 1997, Barr et al., pages 205-212

Adenovirus-based vector delivery to hepatocytes: Rat hepatocytes 1×10¹¹ PFU in 100 g animal.

In dogs (12-17 kg), portal vein is perfused with about 1.5×10¹¹ PFU/kg gives 1 adenovirus genome copy per diploid copy of host DNA

In rabbits (2-4 kg), 1.5×10¹³ virus particles (about 1.5×10¹¹ PFU) gives 100% hepatocyte transduction; 4×10¹² virus particles give 50-75% transduction.

Yang N-S, et al., 281-296

Gold particle-mediated gene delivery: Transfection of mammalian skin tissue-0.1, 0.5, 1.0 and 2.5 μg of DNA/mg particle gives linear relationship with transgene expression levels.

Nabel, et al., 297-305

Liposome-mediated gene delivery in humans:

Protocol 1: 15 nmol DC-Chol/Dope liposomes combined with 1 μg DNA in 0.7 ml. 0.2 ml of the above mixture is injected into the patient's melanoma nodule. For catheter delivery, 0.6 ml of the solution is delivered into the artery.

Protocol 2: 15 nmol DMRIE/Dope liposomes combined with 5 μg DNA in 1.0 ml.

For direct intra-tumor injections, DNA concentrations range from 3 μg complexed with 4.5 nM DMRIE/Dope to 300 μg complexed with 450 nM DMRIE/Dope.

(3) Roessler, et al. 369-374

Gene Transfer to Synovium:

A range of doses, 10⁹-10¹² adenovirus particles containing therapeutic gene/joint, are used. However, the optimal dose for any particular experimental series needs to be determined empirically, and is dependent on both the properties of the recombinant adenoviral genomic backbone being used as well as the transgene being expressed.

For the indirect approach, a variety of methods are well-established, including utilization of cationic lipid or cationic polymer-based transfection and electroporation.

Any of the above-referenced techniques, can be altered to suit the particular needs of those of ordinary skill in the art. Such modifications are well within the level of skill possessed by ordinary practitioners and do not require undue experimentation. These obvious variations are within the scope of this invention.

### EXAMPLES

In order that this invention be more fully understood, the following examples are set forth. These examples are for the purpose of illustrating some preferred embodiments of this invention, and are not to be construed as limiting the scope of this invention in any way.

### Example 1

### Production of TNFp-AIG Constructs

In order to construct chimeric AIG driven by the enhancer cis elements of the TNF promoter, either in a single or multiple copies of the same region or various regions, identification of the regions of interest responsible for optimal inducible expression of the reporter gene is performed.

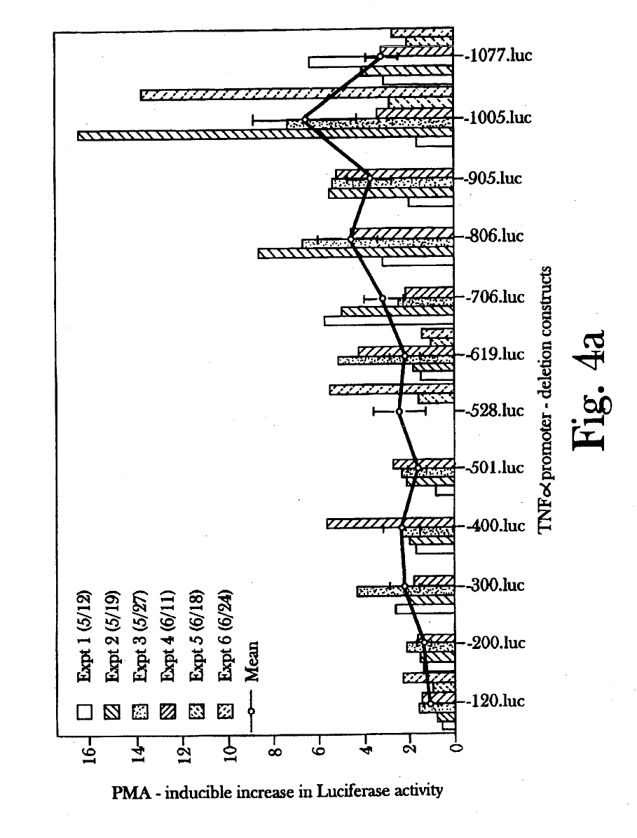

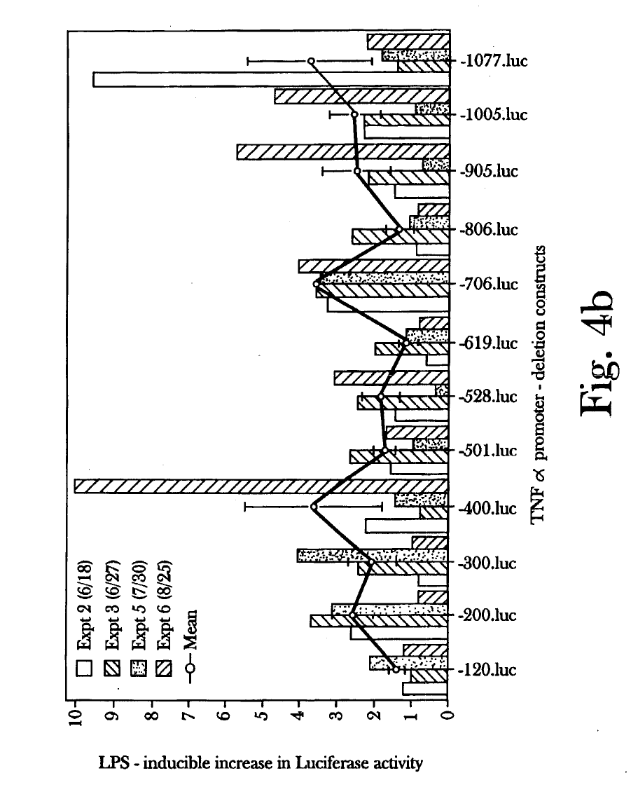

Selection of the TNFα promoter elements for constructing a chimeric gene. The regions of the TNFα promoter are amplified by polymerase chain reaction (PCR) using primers encompassing various deletion constructs of the TNFα promoter (FIG. 3). The regions identified by other investigators in various other cellular systems are used as reference (Rhoades, et al., J. Biol. Chem., 1992, 267, 22102-22107; Leitman, et al., Mol. Cell Biol., 1992, 12, 1352-1356; Pauli U., Crit. Reviews Eukaryotic Gene Expression, 1994, 4, 323-344). The PCR-amplified genes are then cloned upstream of a reporter gene, such as luciferase, in a commercially available promoterless vector. These constructs are tested for their constitutive and inducible expression in various cell lines such as Jurkat (T lymphoblastoid), U973 (myelomonocytic), THP-1 (monocytic), fibroblasts and in vitro cultured human synoviocytes. Identification of the regions responsible for inducible expression of the reporter gene is primarily based on the results obtained using two TNFα-producing cell lines, viz., Jurkat (following stimulation with PMA) and THP-1 (following stimulation with LPS) (FIGS. 4a and 4b). These cells are transiently transfected by using well established methods and commercially available reagents, e.g., DEAE dextran and Superfect. The cis-elements of the TNFα promoter that are responsible for inducible expression of the reporter gene are then used for constructing TNFp-AIG chimeric genes.

Construction of TNFp-AIG chimeric genes. Of the apoptosis inducing genes described herein, the following genes are preferred:

i) cysteine protease—CPP32 (also known as Yama, apopain or caspase 3) and

ii) cysteine protease—Tx/Ty (caspase 4/caspase 5)

The AIGs are used as “prodomain-deleted” truncations in order to potentially augment autocatalysis of caspases. This is essential for conversion of inactive caspase to active form.

Prodomain-deleted CPP32 is amplified using primers corresponding to codons 29-36 and 271-278 (278 is a stop codon). The truncated form of CPP32 is referred to as “αCPP32” or “AIG.1” herein.

For PCR amplification for prodomain-deleted Ty, primers corresponding to the sequences in the Ty gene are synthesized. All caspases discovered so far have homology to the other members of the caspase family. The 3′ primer corresponding to the codons 359-365 (codon 365 is a stop codon) shares 100% sequence homology to the codons 372-378 (codon 378 is a stop codon) in the Tx gene. However, the 5′ primer corresponding to codons 81-87 in the Ty gene does not share 100% homology with the corresponding region in the Tx gene (Tx codons 94-100). Residue 87 (Alanine) in the Ty gene differs from residue 100 (Glycine) in the Tx gene. The PCR amplified product generated from cDNA prepared from activated human peripheral blood lymphocytes possesses the sequence of Tx, due to apparent abundance of Tx transcripts. Therefore, the truncated form of the AIG generated using synthetics oligonucleotide primers corresponding to the sequences in Ty, indeed matches sequences in Tx, albeit flanked by Ty sequences of the primers. The Ty sequences of the primers used also match with the sequence of Tx, except for one codon. Thus the gene used in this invention matches truncated Tx gene with residue G100 to A change. This gene is referred to as “ΔTy/x” or “AIG.2” herein.

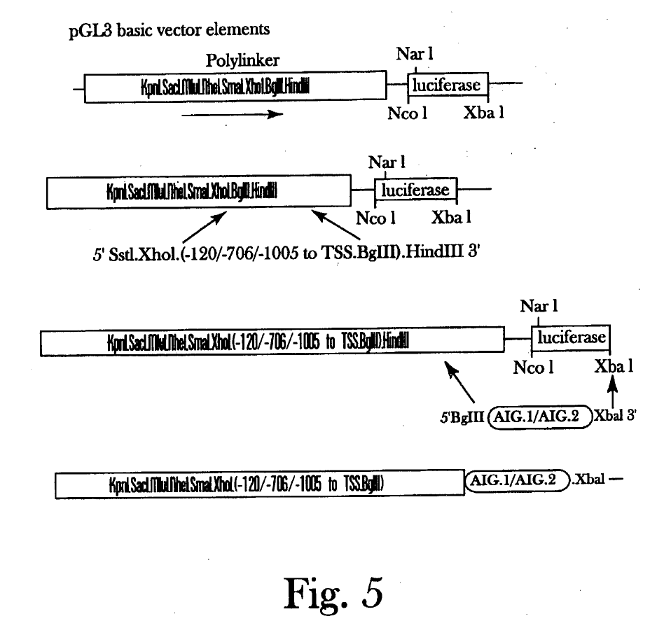

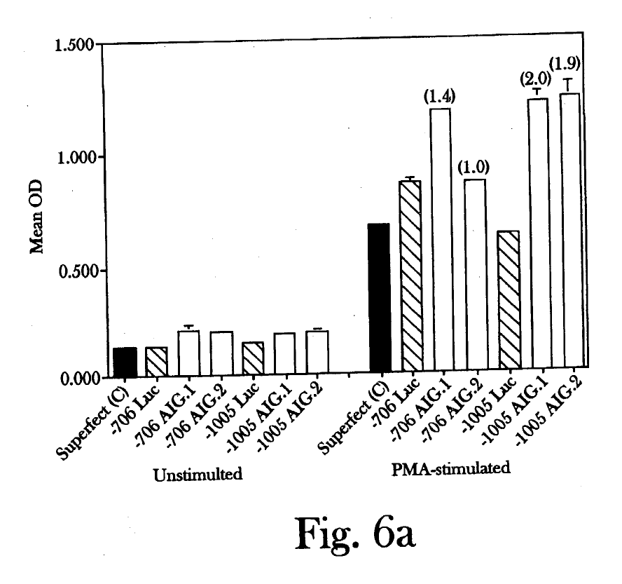

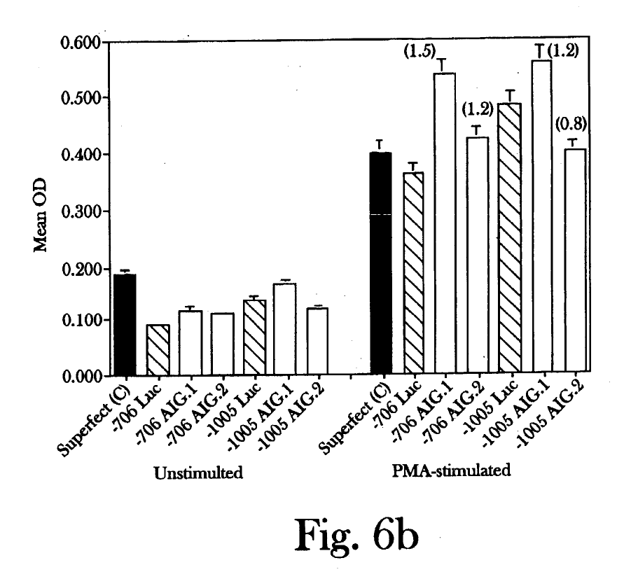

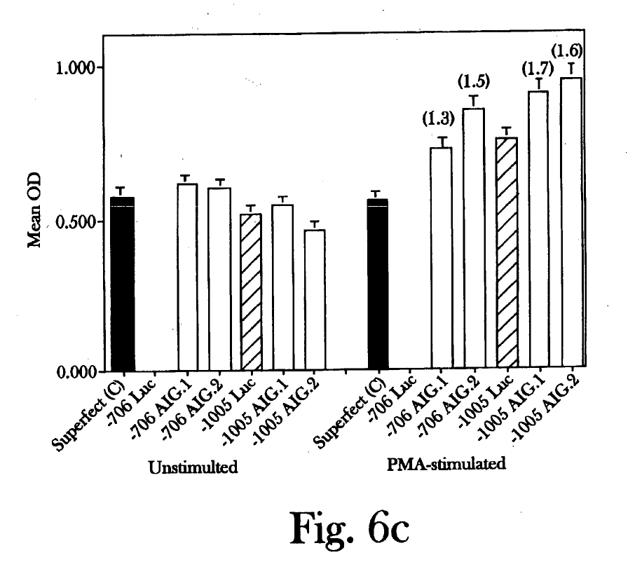

AIG.1 and AIG.2 are inserted downstream of the TNFα promoter by replacing the luciferase reporter gene in deletion constructs (−120, −706 and -1005) of the TNFα promoter (FIG. 5). These constructs are tested for the induction of apoptosis following stimulation of transiently-infected Jurkat and THP-1 cells (FIGS. 6a, b, and c).

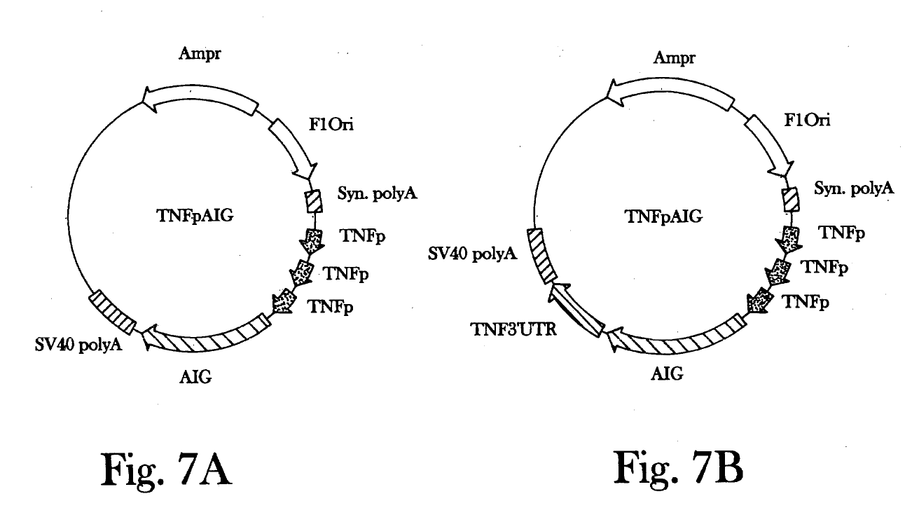

Construction of TNFα super-promoter-AIG chimeric genes. Two broad preferred regions, viz., ER1 (−1005 to −905) (SEQ. ID 10) and ER2 (−706 to −517) (SEQ ID NO: 11) of the TNFα promoter, containing elements responsible for inducible expression of the reporter gene described above (FIGS. 4a and 4b) are PCR amplified and are ligated upstream of the minimal native promoter (−120 through TSS, SEQ ID NO: 3), either as a single copy or multiple copies. Two more regions of SEQ ID NO: 1 and SEQ ID NO: 2 ((−234 to −120) and (−234 to −65)) of the TNFα promoter are also identified as potential enhancer region 3 (ER3) and enhancer region 4 (ER4), respectively, which can be employed in the chimeric constructs using the strategies described below. The super promoter contains multiple (2-10) cassettes of the above mentioned regions containing inducible promoter elements (FIG. 7). This is achieved by PCR amplifying the regions of interest using primers synthesized with restriction sites inserted at the 5′ end of each of the primers. These unique restriction sites flank the amplified gene product of interest. Preferably, PCR amplified AIG is cloned downstream of the TNF super-promoter, replacing the luciferase reporter gene in the original construct as described (FIG. 5) for the native TNFα promoter.

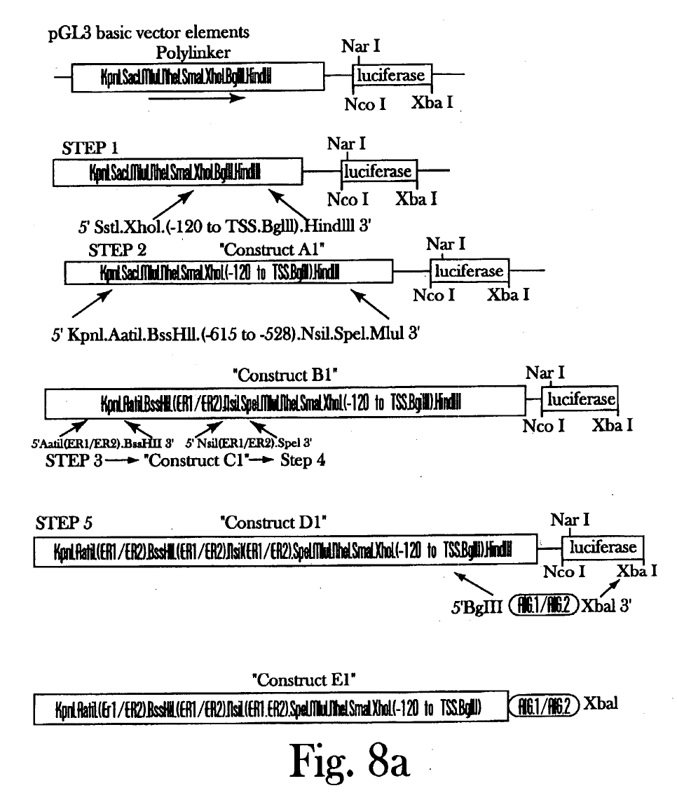

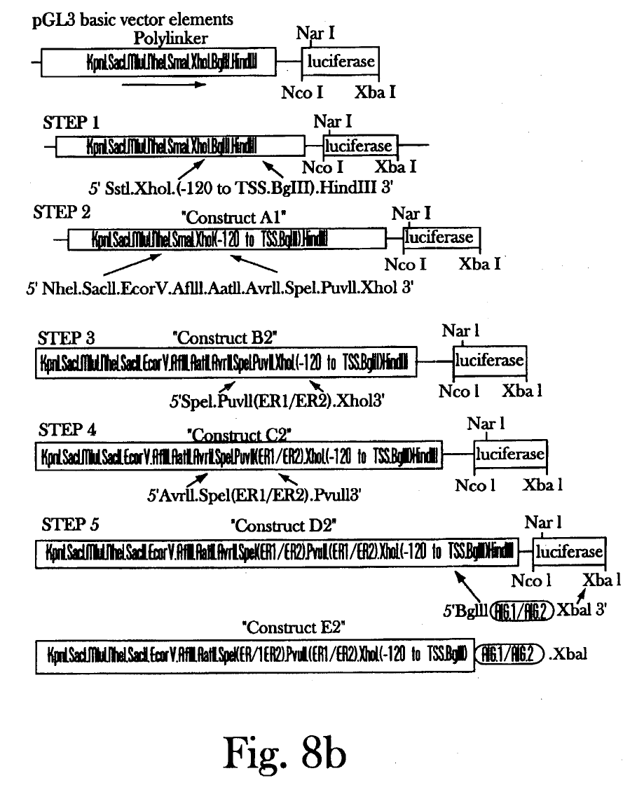

The schemes for construction of a TNFα super-promoter and the linker sequences representing unique restriction sites (these restriction sites are absent in the selected elements of the TNFα promoter and the AIG in question) for efficient directional insertion is outlined below and depicted in FIG. 8:

Scheme 1:

STEP 1: Insertion of the TNFα minimal promoter (−120 to TSS) into the pGL3 basic (promoterless) luciferase vector (Promega):

The elements of pGL3 basic vectors that are used for construction of the chimeric gene TNFp-AIG are shown below.

_KpnI.SacI.MluI.NheI.SmaI.XhoI.BgIII.HindIII.[luciferase].XbaI_{—}

The minimal promoter is PCR amplified using primers containing XhoI and BgIII.HindIII sites, so that XhoI is at the 5′ end and BgIII.HindIII sites are at the 3′ end of the amplified product. This fragment is inserted into the polylinker of the pGL3 basic vector using these same restriction sites. This construct is referred to as “Construct A1” and is as follows:

_KpnI.SacI.MluI.NheI.SmaI.XhoI.(−120 to TSSBglII).HindIII.[luciferase].XbaI_{—}

STEP 2: The enhancer fragment (ER1 or ER2) is PCR amplified using the primer containing several restriction sites. The resulting fragment will have restriction sites KpnI.AatII.BssHII at the 5′ end and NsiI.SpeI.MluI at the 3′ end as follows: 5′ KpnI.AatII.BssHII.(ER1 or ER2).NsiI.SpeI.MluI 3″. The fragment is inserted into the “Construct A1” generated in STEP 1 using KpnI and MluI restriction sites. This construct is referred to as “Construct B1” and is as follows:

_KpnI.AatII.BssHII.(ER1 or ER2).NsiI.SpeI.MluI.NheI.SmaI.XhoI(−120 to TSS BglII).HindIII.[luciferase].XbaI_{—}

STEP 3: The TNFα enhancer fragment (ER1 or ER2)) is amplified using the primers containing restriction sites AatII and BssHII to generate the PCR product as follows: 5′ AatII.(ER1 or ER2).BssHII 3′. This fragment is cloned into the “Construct B1” using these same restriction sites. This construct is referred to as “Construct C1” and is as follows:

_KpnI.AatII.(ER1 or ER2).BssHII.(ER1 or ER2).NsiI.SpeI.MluI.NheI SmaI.XhoI(−120 to TSS BglII).HindIII.[luciferase].XbaI_

STEP 4: The TNFα enhancer fragment (ER1 or ER2) is amplified using the primers containing restriction sites NsiI and SpeI to generate the PCR product as follows: 5′ NsiI.(ER1 or ER2).SpeI 3′. This fragment will be cloned into the “Construct C1” using these same restriction sites. This construct is referred to as “Construct D1” and is as follows:

_KpnI.AatII.(ER1 or ER2).BssHII.(ER1 or ER2).NsiI.(ER1 or ER2). SpeI.MluI.NheI SmaI.XhoI(−120 to TSSBglII).HindIII.[luciferase].XbaI

STEP 5: AIG.1 or AIG.2 (preferred but not limited to AIG.1 and AIG.2; any AIG from the list can be used) coding regions are PCR-amplified using the primers containing BglII and XbaI restriction sites generating the fragment as follows: 5′ BglII.(AIG.1 or AIG.2).XbaI 3″. This fragment is inserted into the “Construct D1” using these same restriction sites. The resulting construct is referred to as “Construct E1” and is as follows:

_KpnI.AatII.(ER1 or ER2).BssHII.(ER1 or ER2).NsiI.(ER1 or ER2).SpeI.MluI.NheI.SmaI.XhoI(−120 to TSS.BglII)[AIG.1 or AIG.2].XbaI_{—}

Alternatively scheme 2 is followed:

Scheme 2:

STEP 1: Same as in scheme I giving rise to “Construct A1”, which is as follows:

KpnI.SacI.MluI.NheI.SmaI.XhoI.(−120 to TSS BglII).HindIII.[luciferase].XbaI

STEP 2: Insertion of additional MCS.

Two complementary oligonucleotides (5′ phosphorylated) providing _NheI.SacI.EcorV.AflII.AatII.AvrII.SpeI.PvuII.XhoI_are synthesized using commercial sources. These oligonucleotides are annealed and then cloned into NheI and XhoI sites of the “Construct A1”. The resulting construct referred to as “Construct B2” and it is as follows:

_KpnI.SacI.MluI.NheI.SacII.EcorV.AflII.AatII.AvrII.SpeI.PvuII.XhoI.(−120 to TSS BglII).HindIII.[luciferase].XbaI_{—}

STEP 3: The TNFα enhancer fragment (ER1 or ER2) is amplified using the primers containing restriction sites SpeI.PvuII at the 5′ end, and XhoI at the 3′ end to generate the PCR product as follows: 5′ SpeI.PvuII.(ER1 or ER2). XhoI 3′. This fragment is cloned into the “Construct B2” using SpeI and XhoI restriction sites. This construct is referred to as “Construct C2” and is as follows:

_KpnI.SacI.MluI.NheI.SacII.EcorV.AflII.AatII.AvrII.SpeI.PvuII.(ER1 or ER2) XhoI.(−120 to TSSBglII).HindIII.[luciferase].XbaI_{—}

STEP 4: The TNFα enhancer fragment (ER1 or ER2) is amplified using the primers containing restriction sites AvrII. SpeI at the 5′ end, and PvuII at the 3′ end to generate the PCR product as follows: 5′ AvrII.SpeI.(ER1 or ER2).PvuII 3′. This fragment is cloned into the “Construct C2” using AvrII and PvuII restriction sites. This construct is referred to as “Construct D2” and is as follows:

_KpnI.SacI.MluI.NheI.SacII.EcorV.AflII.AatII.AvrII.SpeI.(ER1 or ER2) PvuII. (ER1 or ER2) XhoI.(−120 to TSSBglII).HindIII.[luciferase].XbaI_{—}

Thus, using this strategy at least seven copies of the enhancer regions (ER1, ER2 or ER3, individually or in combination), one at a time, can be added by using one more restriction site upstream of the previous one in PCR amplification of the enhancer regions of choice.

Once the desired number of copies of the enhancer regions are added, AIG is inserted downstream of the super-promoter as described in the STEP 5 of the scheme 1.

The inducible expression of the chimeric TNFp-AIG gene is tested by transient transfection of the cell lines mentioned above. The expression of TNFp-AIG gene is measured by detecting apoptosis of transfected cells, assessing AIG expressed proteins in Western blots using commercially available antibodies and assessing protease activity using commercially available, well-documented specific synthetic tetrapeptide substrate.

The inducible expression of the chimeric TNFp-FasL gene is tested by transient transfection of the same cell lines. The cell surface expression of FasL by the transfected cells is quantitated using anti-FasL antibody binding as detected by indirect immunofluorescence and by measuring induction of apoptosis of Fas positive cells.

Regulation of the TNFp-driven expression of a reporter gene. The 3′ untranslated region of the TNFα gene plays an important role in regulation of the TNFα biosynthesis. It is involved in translational expression of the TNFα gene in normal, non-activated states. Importantly, these elements allow de-repression to occur when TNFα-producing cells are activated by external stimuli (Han, J., et al., J. Immunology, 1991, 146, 1843-1848; Crawford, F. K., et al., J. Biol. Chem., 1996, 271, 22383-22390).

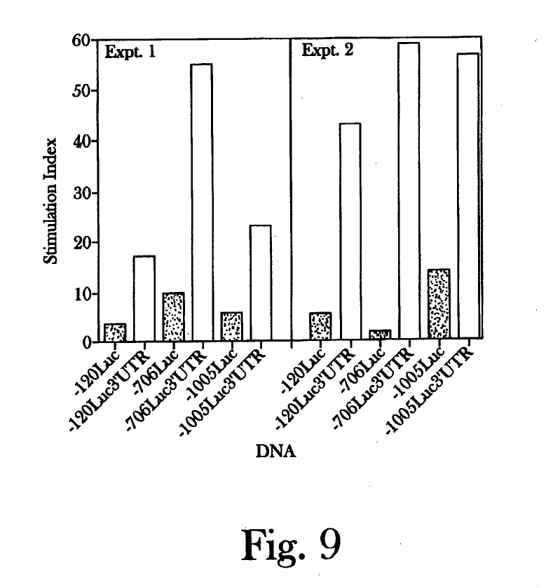

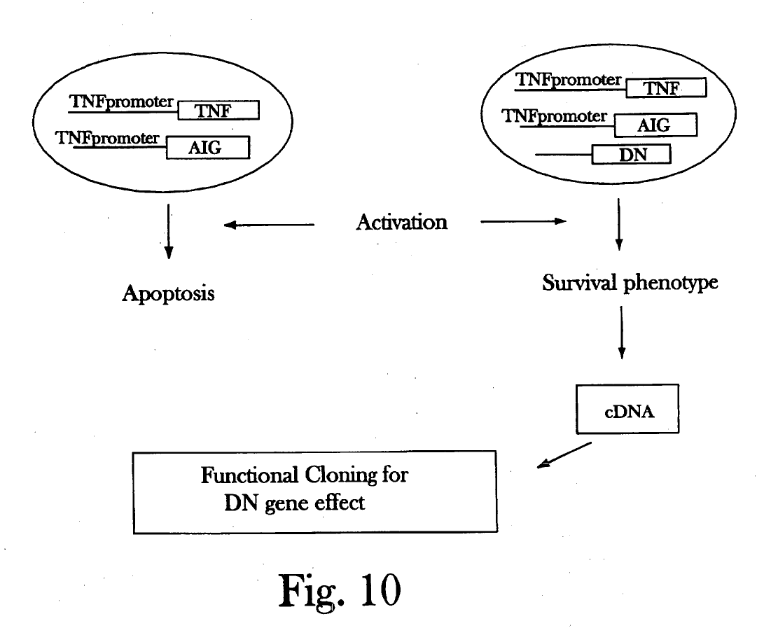

Genetic constructs are made in which the entire 3′ untranslated region (SEQ ID NO: 13) is inserted downstream of the luciferase gene driven by deletion fragments, viz., −120, −706 and −1005 of the TNFα promoter. The results of the transient expression of these constructs are summarized in FIG. 9.

### Example 2

### Testing Protocols

In Vitro Methods:

Luciferase assay: Luciferase activity is determined using commercially available reagents (Promega).

AIG.1 and AIG.2 gene expression:

a) Western blots of the transfected cell lysates are developed using anti-CPP32 antibody as well as anti-PRAP antibody. Anti-PRAP antibody detects both hydrolyzed as well as non-hydrolyzed products of PRAP as an enzymatic action of CPP32.

b) CPP32 enzyme assay: This assay detects enzymatic reaction of CPP32 and breakdown of colorimetric or fluorogenic substrate. A commercially available (Clonotech, Pharmingen) kit is used for this assay.

c) Apoptosis of transfected cells: Apoptosis of transfected cells due to AIG.1 and AIG.2 is determined by staining nuclei by propidium iodide (Krishan, A., J. Cell Biol., 66, 1994, 188-193) and by commercially available Cell Death Elisa kit (Boehringer Mannheim).

Animal Models

Rabbit model of IL-1-induced arthritis (Pettipher E. R., et al., Proc. Natl. Acad. Sci., 1986, 83, 8749-8753): IL-1 is injected into the knee joints of New Zealand White rabbits. Intra-articular injection of IL-1 causes dose-dependent infiltration of leukocytes into the joint space and loss of proteoglycan from the articular cartilage.

Antigen-Induced arthritis: Intra-articular injection of antigen (ovalbumin) into knee joints induces leukocyte accumulation and cartilage degradation that closely resembles rheumatoid arthritis in humans. The joint swelling following the injection was sustained for 14 days.

SCID mice-human synoviocytes model (Houri J. M., et al. Current Opinions in Rheumatol., 1995, 7, 201-205; Sack U., et al., J. Autoimmunity, 1995, 9, 51-58; Geiler T., et al. Arthritis & Rheumatism, 1994, 37, 1664-1671): These are recently developed models for arthritis in which fresh synovial tissue from RA patients is implanted with normal human cartilage into SCID mice either subcutaneously, under the renal capsule (Geiler T., et al., Arthritis & Rheumatism, 1994, 37, 1664-1671), or into knee joints (Sack U., et al., J. Autoimmunity, 1995, 9, 51-58). The implants grow with arthritis-like characteristics, including formation of pannus tissue of high cellular density, bone and cartilage erosion, development of multinuclear giant cells, and invasion of cartilage by synovial fibroblasts.

Indirect Method: Synoviocytes are transfected in vitro with the therapeutic gene and transplanted back in rabbits. Arthritis is induced in these rabbits by injecting IL-1 and expression of the therapeutic gene following activation is assessed. Activation-induced expression of the chimeric gene induces apoptosis in transplanted cells.

Direct Method: Intra-articular injection of the chimeric genes. Any of the gene delivery methods described above, including naked plasmid DNA, cationic liposome-mediated delivery can be used. For use of viral vector-based delivery, chimeric genes are cloned in suitable vectors. The vectors are then modified by deleting eukaryotic promoter present in these vectors. Intra-articular injection of the therapeutic genes inserted in appropriate vectors can then be done to assess therapeutic as well as prophylactic efficacy.

### Example 3

### Selection of TNFα Non-Producer Somatic Cell Variants

Cells (THP-1, Jurkat) are stably transfected in vitro with TNFp-AIG chimeric gene. After several cycles of stimulation, which induces apoptosis in the cells expressing the TNFp-AIG gene, surviving cells are then collected. A cDNA library from these cells is constructed, which is used for functional cloning (Legerski R and Peterson C., Nature, 1992, 359, 70-73; Jaattela M., et al., Oncogene, 1995, 10, 2297-2305).

### Example 4

### Identification and Characterization of Dominant Negative (DN) Genes

THP-1 and Jurkat cells stably transfected with TNFp-AIG are subjected to repeated cycles of stimulation to activate expression of TNFp-AIG. The cells, which do not express negative regulatory genes, undergo apoptosis, whereas those expressing dominant negative genes survive. In these surviving cells DN gene products act in-trans with the TNFα promoter, thereby inhibiting its activations to transcribe AIG, ultimately resulting in survival phenotype. cDNA library is constructed using polyadenylated mRNA from these cells. The DN genes which rescue TNFp-AIG-transfected THP-1 or Jurkat cells from apoptosis are identified by functional cloning as described for other genes (Legerski R. and Peterson C., Nature, 1992, 359, 70-73; Jaattela M., et al., Oncogene, 1995, 10, 2297-2305).

### Example 5

### Cleavage of Caspase-3 Substrate by Cells Expressing AIG.1

The caspase-3 selective substrate, Z-DEVD-AFC, was cleaved with J-E6 cells transfected with cDNA encoding AIG.1. J-E6 cells were transfected with a control vector (cDNA) or the vector expressing AIG. 1 (AIG.1). Transfected cells were harvested at various times after transfection. The cells were lysed and the caspase-3 activity, indicative of functionally active expression of AIG.1, was assessed by determining release of AFC from Z-DEVD-AFC.

Caspase 3 activity in AIG.1-transfected, apoptotic cells was assessed by monitoring cleavage of a fluorochrome tagged synthetic substrate Z-DEVD-AFC (Calbiochem, Cambridge Mass.). The cells were washed once with Ca⁺⁺ and Mg⁺⁺-free PBS and lysed in 10 mM Tris-HCl buffer, pH 7.5, containing 10 mM NaH2PO4/NaHPO4, 130 mM NaCl, 1% Triton X100 and 10 mM NaPPi at a concentration of 2×10⁶ cells/ml for 20 min on ice. The lysates were centrifuged at 14000 rpm for 10 min at 4° C. and the supernatants were assessed for caspase 3 activity. The reactions were carried out in a microtiter plate using HEPES buffer (20 mM HEPES Buffer, 10% Glycerol, 2 mM DTT) in a final volume of 200 μl. The substrates, Z-DEVD-AFC or Ac-DEVD-AMC, were added at a final concentration of 30 μM. Following incubation for 1 hr at 37° C., release of free AFC was determined using an emission wavelength of 480 nm and an excitation of 400 nm.

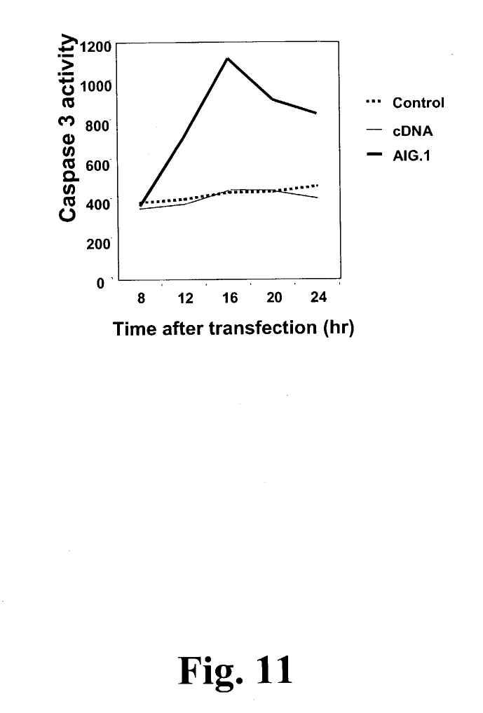

Cleavage of Z-DEVD-AFC, by non-transfected cells or control vector-transfected cells is considered as normal apoptosis. Cleavage of Z-DEVD-AFC more than the control vectors is considered as indicative of supra-normal apoptosis. As shown in FIG. 11, cells expressing AIG.1 cleaved Z-DEVD-AFC at supra-normal apoptosis.

### Example 6

### Cleavage of Poly (ADP-Ribose) Polymerase by Cells Expressing AIG.1

Cleavage of Poly (ADP) ribose polymerase (PARP) is one of the earliest detectable proteolytic event that occurs following high molecular weight fragmentation of chromatin DNA, but before inter-nucleosomal DNA fragmentation in cells undergoing apoptosis. PARP can be cleaved by almost all caspases in vitro; however, in vivo it is primarily targeted by caspases-3 and 7. There are four motifs with a potential cleavage site for caspase-3 in human PARP, namely, DEVD (210-213), DGVD (213-216), DPID (787-790) and DGVD (964-967) (Duriez, P. J. and Shah, G. M. Biochem. Cell Biol. 1997, 75, 337-349). The predominantly used cleavage site is D213 of the DEVD motif, which gives rise to the signature product of 89 kDa in apoptotic cells.

For detection of the PARP cleavage by exogenously expressed AIG.1, HeLa cells were transfected with AIG.1 and a control vector and harvested 24 hrs later for detection of PARP cleavage. The cells were lysed in 50 μl lysis buffer (1% Nonidet P-40, 100 mM Tris pH 7.5, 100 mM NaCl) containing phosphatase inhibitors (50 mM Na₃VO₄, 500 mM EDTA, 100 mM NaF, 100 mM Na₄P₂O₇) and protease inhibitors (0.5% PMSF, aprotinin, 4 μg/ml; antipain, 8 μg/ml and leupeptin, 8 μg/ml). The samples were centrifuged at 14,000 rpm to remove nuclei and supernatants were mixed with equal volume of 2×sample buffer (Novex) containing 10% 2-mercaptoethanol. The samples were boiled at 95° C. for 4 min and 25 μl were subjected to SDS-PAGE using 8-16% Tris-glycine gradient gels (Novex). The resolved proteins were transferred to nitrocellulose membranes (0.2 μm pore size). The nitrocellulose membranes were blocked with 3% BSA/1.5% Ovalbumin (Sigma Chemical Co., St. Louis, Mo.) in TBST (10 mM Tris pH 7.5, 100 mM NaCl, 0.1% Tween 20) overnight at 4° C. to prevent non-specific binding of reagents. The blots were incubated with anti-PARP antibody (Pharmingen, San Diego, Calif., 1:2000 dilution) in 1.5% BSA/TBST for 1 hour at room temperature. The membranes were washed 2 times for 5 min and 2 times for 15 min in TBST and incubated with a 1:2500 dilution of HPR conjugated anti-mouse Ig antibody (Amersham, Piscataway, N.J.) for 1 hour at room temperature. The membranes were washed as described above and exposed to 5 ml ECL chemiluminescence reagents (Amersham, Piscataway, N.J.) for 1 min. Blots were exposed to X-ray film for radiographic detection of the bands.

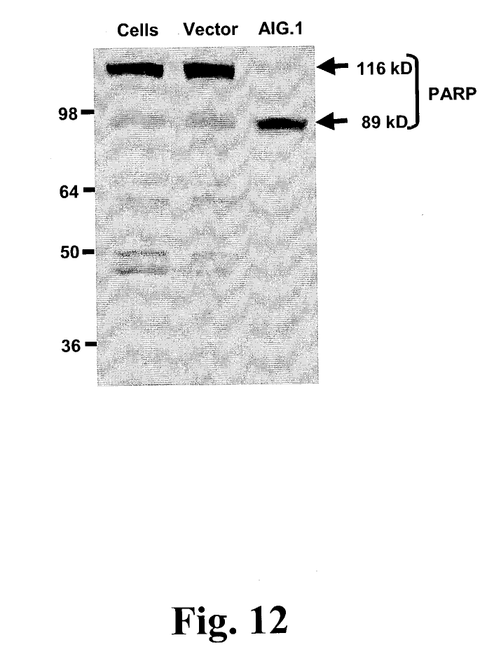

As shown in FIG. 12, only cells expressing AIG.1 underwent apoptosis, as illustrated by cleavage of PARP.

### Example 7

### Induction of Apoptosis in Activated T Cells and Activated Macrophages

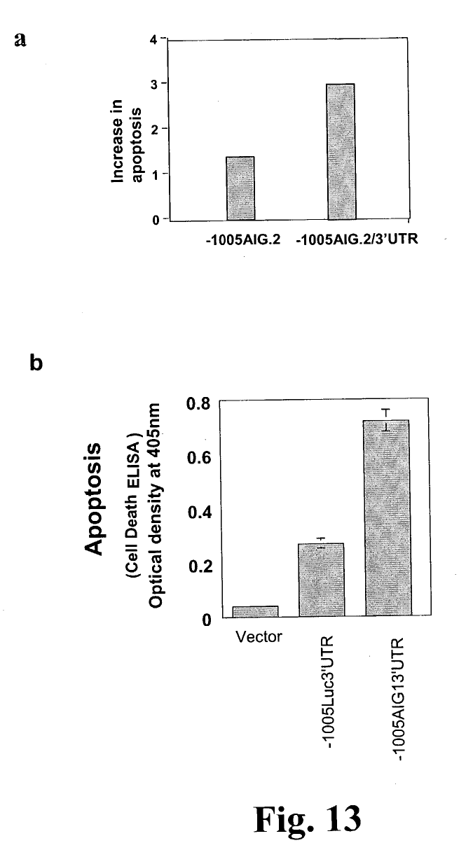

Jurkat (FIG. 13a) or THP-1 cells (FIG. 13b) were transfected with the chimeric genes −1005AIG.1/2 and −1005 AIG.1/2 3′ UTR. The cells were harvested after 24 hours and assessed by cell death ELISA (CDE) to detect DNA fragmentation, indicative of apoptosis fragmentation. Cell Death ELISA (CDE), which measures the fragmented DNA from apoptotic cells, was performed using a kit according to the manufacturer's instructions (Boehringer Mannheim, Indianapolis, Ind.). The cells were washed with phosphate buffered saline (PBS) and lysed in a final volume of 500 μl lysis buffer for 2 hr on ice. The cell lysates containing fragmented DNA in mono- and oligonucleosomes were added to a microtiter plate coated with anti-histone antibody. DNA fragments in the nucleosomes were detected using horseradish peroxidase (HRP) conjugated anti-DNA antibody. The binding of HRP conjugated antibody was assessed by using ABTS (2,2′-azino-di{3-ethlybenzthiazolin-sulfonate(6)}) as a substrate. Optical density was measured using a spectrophotometer (SLT, Spectra, Tecan U.S., Research Triangle Park, N.C.) at 405 nm.

The results are expressed either as absorbance at 405 nm (for THP-1 cells, FIG. 13b) or as a ratio of apoptosis induced by −1005AIG.2 and −1005 AIG.2 3′ UTR to the respective control vectors, where AIG.1/2 was replaced by the luciferase gene (for Jurkat cells, FIG. 13a). Apoptosis induced by control vectors is considered as the background or normal apoptosis. Apoptosis more than that induced by control vectors is considered as supra-normal apoptosis. As shown in FIGS. 13a and 13b, AIG containing vectors induced supra-normal apoptosis when expressed in Jurkat cells (a cell line representative of activated T cells) and THP-1 cells (a cell line representative of activated macrophages), respectively.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

The papers cited herein are expressly incorporated in their entireties by reference.

What is claimed is:

1. A chimeric gene comprising at least one TNFα enhancer attached to a functional copy of a minimal TNFα promoter and further attached to at least one copy of an apoptosis-inducing gene, wherein the expression of the apoptosis-inducing gene is driven by the TNFα promoter, and wherein the chimeric gene is expressed in inflammatory cells producing TNFα such that supra-normal apoptosis is induced.

2. The chimeric gene according to claim 1 comprising two or more copies of the TNFα enhancer.

3. The chimeric gene according to claim 1 wherein the apoptosis-inducing gene encodes a protein selected from the group consisting of caspase-1, caspase-2, caspase-3, caspase-4, caspase-5, caspase-6, caspase-7, caspase-8, caspase-9, caspase-10, Granzyme A, Granzyme B, and Fas ligand.

4. The chimeric gene according to claim 3 wherein the apoptosis-inducing gene encodes a protein selected from the group consisting of caspase 3, caspase 4, caspase 5, and Granzyme B.

5. A chimeric gene comprising 2 to 10 cassettes of a TNFα enhancer attached to at least one copy of a minimal TNFα promoter, and at least one copy of an apoptosis-inducing gene, wherein the apoptosis-inducing gene encodes a protein selected from the group consisting of caspase-3, caspase-4, caspase-5, and Granzyme B, and wherein the expression of the apoptosis-inducing gene is driven by the TNF promoter such that supra-normal apoptosis is induced.

6. A chimeric gene comprising:
(a) at least one enhancer attached to a functional copy of a minimal promoter, wherein the promoter is a promoter for a gene or combination of genes activated in inflammatory cells or in cells at a site of inflammation; and
(b) further attached to at least one copy of an apoptosis-inducing gene, wherein the expression of the apoptosis-inducing gene is driven by the promoter, and
wherein the promoter is a promoter for a gene encoding a protein selected from the group consisting of cytokines, interleukins, cell adhesion molecules, chemokines, pro-inflammatory enzymes, interleukin receptors, cell adhesion molecule ligands, and chemokine receptors; and
wherein the chimeric gene is expressed in inflammatory cells such that supra-normal apoptosis is induced.

7. The chimeric gene according to claim 6 wherein the promoter is a promoter for a gene encoding a protein selected from the group consisting of TNFβ, IL-1α, IL-1β, IL-2, IL-6, IL-8, GM-CSF, and interferony.

8. The chimeric gene according to claim 6 wherein the promoter is a promoter for a gene encoding a protein selected from the group consisting of selecting, integrins, ICAM-1, and V-CAM.

9. The chimeric gene according to claim 6 wherein the promoter is a promoter for a gene encoding a protein selected from the group consisting of MIP-1α, MIP-1β, MCP1-4, RANTES, Mig, NAP2, IP10, and Gro α-γ.

10. The chimeric gene according to claim 6 wherein the promoter is a promoter for a gene encoding a protein selected from the group consisting of COX-2, iNOS, phospholipases, and proteases.

11. A pharmaceutical composition comprising a gene according to claim 1.

12. The pharmaceutical composition comprising a gene according to claim 11 selected from the group consisting of: SEQ ID NO: 4, SEQ ID NO: 5, SEQ ID NO: 6, SEQ ID NO: 7, SEQ ID NO: 8, and SEQ ID NO: 9, wherein the 3′UTR of the TNFα gene of SEQ ID NO: 13 is ligated downstream of the apoptosis-inducing gene.

13. A pharmaceutical composition comprising a gene according to claim 5.

14. A pharmaceutical composition comprising a gene according to claim 6.

15. A method for treating an inflammatory disorder in a patient comprising the step of inducing supra-normal apoptosis in inflammatory cells or cells at a site of inflammation of the patient by introducing into the cells a chimeric gene according to claim 1.

16. The method according to claim 15 wherein the induction of supra-normal apoptosis does not induce an inflammatory response in the patient.

17. The method according to claim 15 wherein the inflammatory cell is a TNFα producing cell.

18. The method according to claim 15 wherein the inflammatory disorder is selected from the group consisting of: rheumatoid arthritis, multiple sclerosis, Guillain-Barre syndrome, Crohn's disease, ulcerative colitis, psoriasis, graft versus host disease, lupus erythematosus, insulin-dependent diabetes mellitus, psoriatic arthritis, sarcoidosis, hypersensitivity pneumonitis, ankylosing spondylitis, Reiter's syndrome, and systemic sclerosis.

19. The method according to claim 18 wherein the inflammatory disorder is rheumatoid arthritis.

20. A method for treating an inflammatory disorder in a patient comprising inducing supra-normal apoptosis in inflammatory cells or cells at the site of inflammation of the patient by introducing into the cells a chimeric gene according to claim 5 without inducing an inflammatory response in the patient.

21. A process for constructing a chimeric gene comprising at least one TNFα promoter enhancer attached to a functional copy of a minimal TNFα promoter and further attached to at least one copy of an apoptosis-inducing gene comprising the steps of:
(a) amplifying a TNFα promoter by a polymerase chain reaction using primers encompassing deletion constructs of the TNFα promoter;
(b) cloning the PCR-amplified genes obtained in step (a) upstream of a reporter gene;
(c) testing the constructs obtained in step (b) for their constitutive and inducible expression in at least one TNFα-producing cell line;
(d) selecting TNFα promoter responsible for inducible expression of the reporter in the cell line; and either
(e) PCR-amplifying TNFα promoter regions that enhance expression of the reporter to obtain an enhancer and ligating at least one copy of the enhancer upstream of the promoter; or
(f) inserting at least one copy of a prodomain-deleted apoptosis-inducing gene downstream of the TNFα promoter by replacing the reporter gene with the apoptosis-inducing gene deletion constructs to obtain a chimeric gene or
(g) PCR-amplifying a TNFα-3′UTR and ligating downstream of the reporter gene, or any combination of these procedures,
wherein the expression of the apoptosis-inducing gene is driven by the TNFα promoter such that supra-normal apoptosis is induced.

22. The process according to claim 21 wherein two or more copies of the enhancer are inserted upstream of the promoter.

23. A process according to claim 21 wherein reporter gene is luciferase.

24. The process according to claim 21 wherein the enhancer comprises SEQ ID NO: 10 or SEQ ID NO: 11.

25. The process according to claim 21 wherein the prodomain-deleted apoptosis-inducing gene is selected from the group consisting of caspase 3, caspase 4, caspase 5, Granzyme B.

26. The process according to claim 21 producing a gene selected from the group consisting of SEQ ID NO: 4, SEQ ID NO: 5, SEQ ID NO: 6, SEQ ID NO: 7, SEQ ID NO: 8, SEQ ID NO: 9.

27. The process according to claim 21 wherein the cell lines for testing constructs are selected from the group consisting of T lymphoblastoid, myelomonocytic, monocytic, fibroblast, and cultured human synoviocytes.


SEQUENCE LISTING (13 sequences)

SEQ ID NO: 1 - DNA, 1178 bp, Homo sapiens
  1 to 1178: full-length human TNFa promoter
ggggaagcaaaggagaagctgagaagatgaaggaaaagtcagggtctggaggggcgggggtcagggagctcctgggagatatggccacatgtagcggctctgaggaatgggttacaggagacctctggggagatgtgaccacagcaatgggtaggagaatgtccagggctatggaagtcgagtatcggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggtctgtgaattcccgggggtgatttcactccccgggctgtcccaggcttgtccctgctacccccacccagcctttcctgaggcctcaagctgccaccaagcccccagctccttctccccgcagacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatncccgcccccgcgatggagaagaaaccgagacagaaggtgcagggcccactaccgcttcctccagatgagcttatgggtttctccaccaaggaagttttccgctggttgaatgattctttccccgccctcctctcgccccagggacatataaaggcagttgttggcacacccagccagcagacgctccctcagcaaggacagcagaggaccagctaagagggagagaagcaactgcagaccccccctgaaaacaaccctcagacgccacatcccctgacaagctgccaggcaggttct

SEQ ID NO: 2 - DNA, 1096 bp, Homo sapiens
  1 to 1096: native human TNFa promoter
gaggccgccagactgctgcaggggaagcaaaggagaagctgagaagatgaaggaaaagtcagggtctggaggggcgggggtcagggagctcctgggagatatggccacatgtagcggctctgaggaatgggttacaggagacctctggggagatgtgaccacagcaatgggtaggagaatgtccagggctatggaagtcgagtatggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctc

SEQ ID NO: 3 - DNA, 139 bp, Homo sapiens
  1 to 139: native minimal TNFa promoter
ccgcttcctccagatgagctcatgggtttctccaccaaggaagttttccgctggttgaatgattctttccccgccctcctctcgccccagggacatataaaggcagttgtatggcacacccgccagcagacgctccctc

SEQ ID NO: 4 - DNA, 904 bp, Artificial sequence
  1 to 139; 140 to 151; 152 to 904: chimeric gene TNFp120 AIG.1.  Residues 1 to 139 comprise the promoter sequence; residues 140 to 151 comprise the linker sequence, and the remaining residues comprise the AIG.1 sequence
ccgcttcctccagatgagctcatgggtttctccaccaaggaagttttccgctggttgaatgattctttccccgccctcctctcgccccagggacatataaaggcagttgttggcacacccagccagcagacgctccctcagcagatccaccatgtctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggctaaccgaaaggtggcaacagaatttgagtccttttcctttgacgctacttttcatgcaaagaaacagattccatgtattgtttccatgctcacaaaagaactctatttttatcactaa

SEQ ID NO: 5 - DNA, 1490 bp, Artificial sequence
  1 to 724; 725 to 736; 736 to 1490: chimeric gene TNFp706 AIG.1.  Residues 1 to 724 comprise the promoter sequence; residues 725 to 736 comprise the linker sequence, and remaining residues comprise the AIG.1 sequence
tccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcagatccaccatgtctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccattgctgaaacagtatgccgacaagcttgaatttatgcacattcttacccgggctaaccgaaaggtggcaacagaatttgagtccttttcctttgacgctacttttcatgcaaagaaacagattccatgtattgtttccatgctcacaaaagaactctatttttatcactaa

SEQ ID NO: 6 - DNA, 1789 bp, Artificial sequence
  1 to 1023; 1024 to 1036; 1037 to 1789: chimeric gene TNFp1005 AIG.1.  Residues 1 to 1023 comprise the promoter sequence; residues 1024 to 1036 comprise the linker sequence, and the remaining residues comprise the AIG.1 sequence
ggcgggggtcagggagctcctgggagatatggccacatgtagcggctctgaggaatgggttacaggagacctctggggagatgtgaccacagcaatgggtaggagaatgtccagggctatggaagtcgagtatggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcagatccaccatgtctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggctaaccgaaaggtggcaacagaatttgagtccttttcctttgacgctacttttcatgcaaagaaacagattccatgtattgtttccatgctcacaaaagaactctatttttatcactaa

SEQ ID NO: 7 - DNA, 1008 bp, Artificial sequence
  1 to 138; 139 to 150; 151 to 1008: chimeric gene TNFp120 AIG.2.  Residues 1 to 138 comprise the promoter sequence; residues 139 to 150 comprise the linker sequence, and the remaining residues comprise the AIG.1 sequence
ccgcttcctccagatgagctcatgggtttctccaccaaggaagttttccgctggttgaatgattctttccccgccctcctctcgccccagggacatataaaggcagttgttggcacacccagccagcagagctccctcagcagatccaccatggctggaccacctgagtcagcagaatctacagatgccctcaagctttgtcctcatgaagaattcctgagactatgtaaagaaagagctgaagagatctacccaataaaggagagaaacaaccgcacacgcctggctctcatcatatgcaatacagagtttgaccatctgcctccgaggaatggagctgactttgacatcacagggatgaaggagctacttgagggtctggactatagtgtagatgtagaagagaatctgacagccagggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctggaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattga

SEQ ID NO: 8 - DNA, 1587 bp, Artificial sequence
  1 to 724; 725 to 736; 737 to 1587: chimeric gene TNFp706 AIG.2.  Residues 1 to 724 comprise the promoter sequence; residues 725 to 736 comprise the linker sequence, and the remaining residues comprise the AIG.1 sequence
tccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccaaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcagatccaccatggctggaccacctgagtcagcagaatctacagatgccctcaagctttgtcctcatgaagaattcctgagactatgtaaagaaagagctgaagagatctacccaataaaggagagaaacaaccgcacacgcctggctctcatcatatgcaatacagagtttgaccatctgcctccgaggaatggagctgacttgacatcacaggatgaaggagtacttgagggtctggactatgtgtagatgtgaagagaatcgacagccaggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctggaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattga

SEQ ID NO: 9 - DNA, 1894 bp, Artificial sequence
  1 to 1024; 1025 to 1036; 1037 to 1894: chimeric gene TNFp1005 AIG.2.  Residues 1 to 1024 comprise the promoter sequence; residues 1025 to 1036 comprise the linker sequence, and the remaining residues comprise the AIG.1 sequence
ggcgggggtcagggagctcctgggagatatggccacatgtagcggctctgaggaatgggttacaggagacctctggggagatgtgaccacagcaatgggtaggagaatgtccagggctatggaagtcgagtatggggacccccccttaacgaagacagggccatgtagagggccccagggagtgaaagagcctccaggacctccaggtatggaatacaggggacgtttaagaagatatggccacacactggggccctgagaagtgagagcttcatgaaaaaaatcagggaccccagagttccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctccttctccccgcagggacccaaacacaggcctcaggactcaacacagcttttccctccaaccccgttttctctccctcaaggactcagctttctgaagcccctcccagttctagttctatctttttcctgcatcctgtctggaagttagaaggaaacagaccacagacctggtccccaaaagaaatggaggcaataggttttgaggggcatggggacggggttcagcctccagggtcctacacacaaatcagtcagtggcccagaagacccccctcggaatcggagcagggaggatggggagtgtgaggggtatccttgatgcttgtgtgtccccaactttccaaatccccgcccccgcgatggagaagaaaccgagacagaaggtgcagggcccactaccgcttcctccagatgagctcatgggtttctccaccaaggaagttttccgctggttgaatgattctttccccgccctcctctcgccccagggacatataaaggcagttgttggcacacccagccagcagacgctccctcagcagatccaccatggctggaccacctgagtcagcagaatctacagatgccctcaagctttgtcctcatgaagaattcctgagactatgtaaagaaagagctgaagagatctacccaataaaggagagaaacaaccgcacacgcctggctctcatcatatgcaatacagagtttgaccatctgcctccgaggaatggagctgactttgacatcacagggatgaaggagctacttgagggtctggactatagtgtagatgtagaagagaatctgacagccagggatatggagtcagcgctgagggcatttgctaccagaccagagcacaagtcctctgacagcacattcttggtactcatgtctcatggcatcctggagggaatctgcggaactgtgcatgatgagaaaaaaccagatgtgctgctttatgacaccatcttccagatattcaacaaccgcaactgcctcagtctgaaggacaaacccaaggtcatcattgtccaggcctgcagaggtgcaaaccgtggggaactgtgggtcagagactctccagcatccttggaagtggcctcttcacagtcatctgagaacctggaggaagatgctgtttacaagacccacgtggagaaggacttcattgctttctgctcttcaacgccacacaacgtgtcctggagagacagcacaatgggctctatcttcatcacacaactcatcacatgcttccagaaatattcttggtgctgccacctagaggaagtatttcggaaggtacagcaatcatttgaaactccaagggccaaagctcaaatgcccaccatagaacgactgtccatgacaagatatttctacctctttcctggcaattga

SEQ ID NO: 10 - DNA, 123 bp, Homo sapiens
  1 to 123: TNFa promoter enhancer region 1 (ER1)
ggggcgggggtcagggagctcctgggagatatggccacatgtagcggctctgaggaatgggttacaggagacctctggggagatgtgaccacagcaatgggtaggagaatgtccagggctatg

SEQ ID NO: 11 - DNA, 190 bp, Homo sapiens
  1 to 190: TNFa promoter enhancer region 2 (ER2)
tccttggaagccaagactgaaaccagcattatgagtctccgggtcagaatgaaagaagaaggcctgccccagtggggtctgtgaattcccgggggtgatttcactccccggggctgtcccaggcttgtccctgctacccccacccagcctttcctgaggcctcaagcctgccaccaagcccccagctcct

SEQ ID NO: 12 - DNA, 223 bp, Artificial sequence
  1 to 223: genetically engineered multiple cloning sites genetically engineered upstream of the minimal TNF promoter in the -120pGL3 construct
ggtaccgagctcttacgcgtgctagccgcggatatcttaagacgtcctaggactagtcagctgctcgagccgcttcctccagatgagctcatgggtttctccaccaaggaagttttccgctggttgaatgattctttccccgccctcctctcgccccagggacatataaaggcagttgttggcacacccagccagcagacgctccctcagcagatctaagctt

SEQ ID NO: 13 - DNA, 787 bp, Homo sapiens
  1 to 787: TNFa 3′ untranslated region
tctagaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagatctaga